(19) United States
(12) Patent Application Publication
(10) Pub. No.: US 2010/0168769 A1
(43) Pub. Date: Jul. 1, 2010

(54) LAPAROSCOPIC KNOT PUSHING DEVICE

(75) Inventors: Richard S. Wilkenfeld, Montgomery, TX (US); Rhoda Wilkenfeld, Boca Raton, FL (US)

Correspondence Address: BUSKOP LAW GROUP, P.C., 4511 Dacoma Street, HOUSTON, TX, 77092, US

(73) Assignee: Richard S. Wilkenfeld, Conroe, TX (US)

(21) Appl. No.: 12/346,046

(22) Filed: Dec. 30, 2008

(51) Int. Cl. A61B 17/062

(52) U.S. Cl. 606/148

(57) ABSTRACT

A laparoscopic knot pushing device having an elongated body with a first end adapted for gripping by a doctor, a planar face formed in the second end. The planar face is opposite the elongated body, and has a first side with a first rounded edge and a second rounded edge and the planar face is positioned 5 degrees to 20 degrees from the axis of the elongated body. The device further comprises a hole with a top and bottom rounded edges are disposed in the planar face aligned with the axis to the elongated body axis.

### FIELD

The present embodiments relate to a laparoscopic knot pushing device used in suturing patients, particularly lap-band patients.

### BACKGROUND

A need exists for an easy to use tool that can be inserted in a narrow catheter or trocar for pushing throws for forming knots down into a patient to tie off a suture stitch.

A further need exists for an easy to manipulate, easy to sterilize and easy to manufacture tool for sliding knots that are not slip knots into patients.

The present embodiments meet these needs.

### BRIEF DESCRIPTION OF THE DRAWINGS

The detailed description will be better understood in conjunction with the accompanying drawings as follows:

FIG. 1 is a top view of an embodiment of the laparoscopic knot pushing device.

FIG. 2 is a detail of the planar face and hole of the laparoscopic knot tying device.

FIG. 3 shows a detail of a hole in the planar face.

FIG. 4 depicts a camera and targeting light mounted to the elongated body of the invention.

The present embodiments are detailed below with reference to the listed Figures.

### DETAILED DESCRIPTION OF THE EMBODIMENTS

Before explaining the present apparatus in detail, it is to be understood that the apparatus is not limited to the particular embodiments and that it can be practiced or carried out in various ways.

The present embodiments relate to a laparoscopic knot pushing device having an elongated body, a planar face and a hole for use in suturing patients, particularly lap-band patients or any other minimally invasive surgery procedure.

A benefit of this invention is that with the knot pushing device, suturing becomes more secure than a prepared knot or a slip knot. This device provides secure knots, known as surgical knots. Surgical knots prevent tissue from becoming disengaged from the suture and each other. The use of surgical knots enables faster healing by a patient because the tissue contact is maintained.

Another benefit of this invention is that the user can firmly grasp the device, and the device provides a “non-slip” feature for the user. When a doctor can hold the knot tying device securely, damage to adjacent tissue is prevented.

A benefit of the invention is that in the embodiment wherein the device can be made from sterilizable material, the device can be stiff, but slightly flexible for again allowing a secure knot to be inserted with a tight knot while simultaneously preventing the suture from breaking.

Still another benefit of the invention is that the device is easier to use and provides more secure knots than intracorporal knot tying. Sewing inside a body laparoscopically, with two needle holders is complex and difficult. This device can allow interbody sewing in part, to occur outside of the body, wherein a knot is formed outside of the body and then the device slides the knot into the body, which is less complicated sewing than trying to tie a knot in the body itself. This device assists in sewing by providing a device for knot tying.

The device allows a surgeon to make knots in a larger volume of space outside of the body. The device further allows the surgeons working in very small spaces to make secure surgical knots quickly, easily with less stress.

Another benefit of the invention is that with the device, it takes less time to suture a patient than suturing without the knot tying device. The knot tying device makes surgery easier on a patient and enables the patient to spend less time under anesthesia. As an example, in a surgery the device and method for knot tying can save up to about 20 percent of the time for suturing by enabling the surgeon to tie knots outside of the body and then slip them into the body.

Another benefit of the invention is that the device enables a greater range of sutures to be used on a patient. This device further allows increased versatility for a surgeon in the doctor's selection of sutures for a particular procedure. This device is particularly good for increasing versatility in the use of different types of sutures for laparoscopic surgeries. Now, sutures made of nylon, dissolving sutures, Dacron™ sutures, Neuralon™ sutures, Ethabond™ sutures and silk sutures can be easily used.

The device can enable a physician to perform surgeries on a patient faster than traditional surgeries, by at least 10 percent, which can reduce or eliminate diabetes in the patient.

The device can further enable a surgeon, to perform surgeries on a patient faster, than without the device, reducing the chance of tissues coming apart, where the surgeries are to reduce or eliminate high blood pressure in a patient undergoing lap band surgery.

The device can further enable a surgeon to repair hiatal hernias using native or synthetic tissues with less difficulty and greater security than non-suturing repairs such as tacking devices.

The device can enable a surgeon to perform surgeries on a patient to reduce or eliminate acid reflux very inexpensively, with less than about ½ the cost of performing the same surgery with a tacking device.

The device can be used on humans, horses, cows, or other mammals over about 15 pounds.

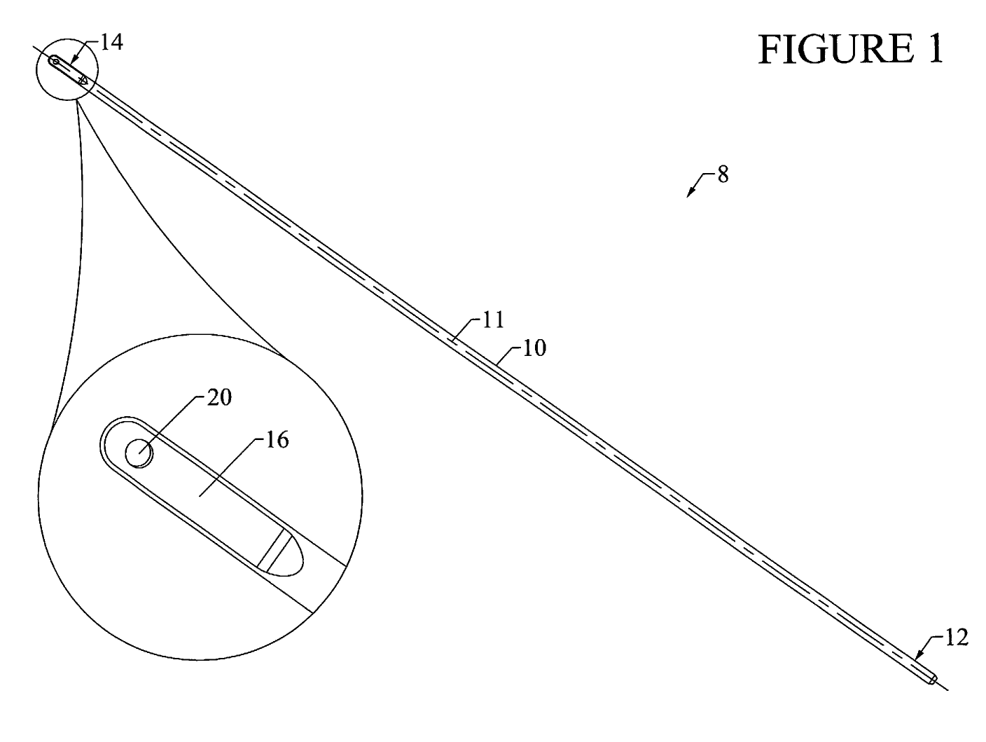

Turning now to FIG. 1, the laparoscopic knot pushing device (8) has an elongated body (10) having an elongated body axis (11).

The elongated body can be between about 18 inches to about 24 inches long and have an overall diameter ranging from between about 3 millimeters to about 9.5 millimeters. The elongated body can have a planar face (16) with a hole (20). The elongated body is showing having a first end (12) for holding by a surgeon and a second end (14) with unique features.

In this embodiment, the elongated body can be made of stainless steel, or a sterilizable, non-deformable, impact resistant polymer, such as a polypropylene copolymer with polyethylene or a polypropylene with some polyvinyl chloride blended in. In another embodiment, the elongated body can be made of a stiff material having some flexibility such as a graphite composite or a polymer with between about 2 weight percent to about 20 weight percent of an elastomeric material blended in.

The elongated body must be sterilizable, such as with an autoclave or with ultraviolet light or sterilizable with chemicals and resist degradation during sterilization.

An embodiment of the device can be reused by a surgeon. A hollow embodiment can be reused as well as a solid version many times, particularly if both embodiments are made of a surgical grade metal.

Another embodiment of the device, contemplates that a hollow, throw-away version of the device can be used, that is, a disposable device. A solid elongated body can be used in a disposable version as well. Disposable versions can have the advantage of being lightweight, easy to ship, and easy to store.

The elongated body can have an integral planar face or a removable planar face. The device can be an integral one-piece unit formed from a single piece of steel, such as surgical grade stainless steel.

The elongated body is can have a diameter that fits within standard sized trocars.

If the device is a two-piece structure, the planar face can threadably engaged with the elongated body, or it can be force fit into the body, such as a cavity formed in the end of the body.

The elongated body can also be made of polycarbonate or another autoclavable or sterilizable polymer that does not deform at high temperatures in an autoclave.

An embodiment contemplates that the device can be about 100 percent metal, about 100 percent polymer or can be an autoclavable non-deformable polymer disposed over a metal cylindrical body.

An embodiment of the invention contemplates that the elongated body can have a solid elongated body about 19 inches long, with body diameter of about 5 millimeters and a hole diameter of about 3 millimeters.

The shape of the elongated body can be rounded. The elongated body can be a rounded 8 sided structure, a rounded 4 sided structure, a rounded 6 sided structure, a cylinder structure or a conical structure.

The elongated body first end (12) can be adapted for gripping by a user, such as a lap band surgeon or another laparoscopic surgeon.

The device can be used for tying a knot after a suture is placed and pushing the knot down into a body through a cylindrical tube, such as a trocar. Trocars can vary in diameter from about 5 millimeters to about 15 millimeters and the device can have different diameters to fit within the different sized trocars. Sutures and the completed knot can then be used to connect patient tissue securely with this device.

FIG. 1 shows planar face (16) can be formed in the second end (14). The planar face can be integral with the elongated body (10). The planar face (16) has a hole (20) in line with the axis (11) of the elongated body, but can be formed perpendicular to the second end (14) axis of the body.

In an embodiment, the hole can be formed perpendicular to the axis of the elongated body or perpendicular to the second end (14).

The planar face can be tapered and can have a thickness ranging between about 2 millimeters to about 4 millimeters. However, if a larger diameter elongated body is used, the planar face can taper from about 2 millimeters to about 8 millimeters when the elongated body diameter is about 9 millimeters.

The width of the planar face can be up to about 9 millimeters. The hole in the planar face can have a diameter ranging from about 1.5 millimeters to about 7 millimeters. The elongated body can be tapered for a larger diameter at the first end to a smaller diameter by about 50 percent at the second end. The planar face can taper from the body that can be tapered to a diameter between about 20 percent to about 80 percent less than the diameter of the elongated body.

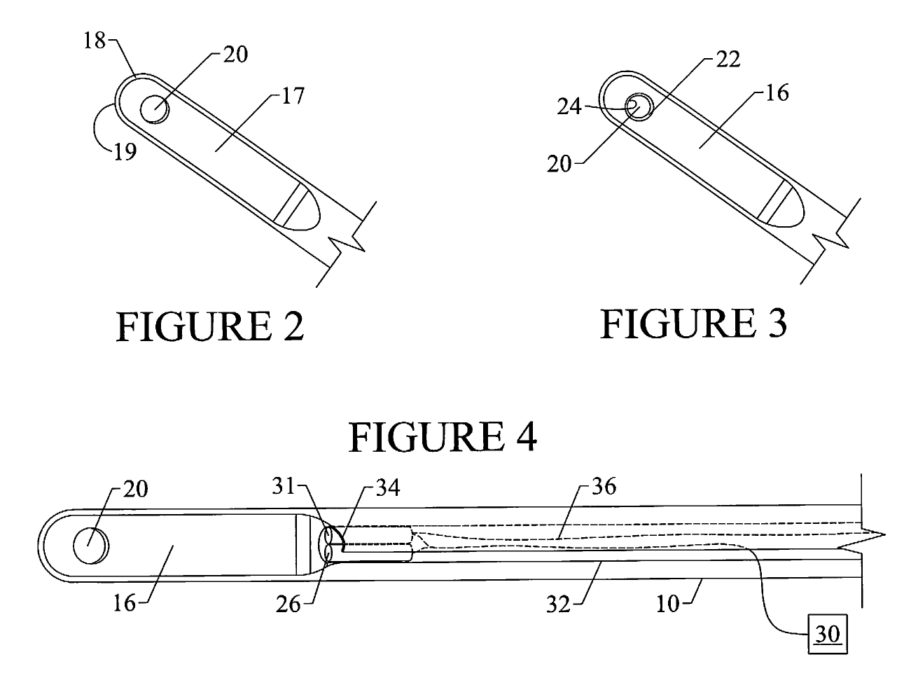

FIG. 2 shows a detail of the planar face having a first side (17) with a first rounded edge (18), a second rounded edge (19) opposite the first rounded edge, and the hole (20). Angled faces can cut the suture material, making it impossible to slide the newly tied knot into the body smoothly, so they are avoided with this device.

The planar face, in an embodiment can have the first and second rounded edges on at least a portion of the first side, that is the entire first side does not have to have rounded edges, just the distal tip of the device.

The planar face can be positioned from about 5 degrees to about 20 degrees from the axis of the elongated body (see FIG. 4). In an embodiment of the invention, the planar face can be between about 8 degrees to about 10 degrees from the axis of the elongated body.

FIG. 3 shows a detail of the hole (20) with a top rounded edge (22) on the inside of the hole and a bottom rounded edge (24) on the inside of the hole disposed in the planar face (16). The hole can have a diameter from about 1.5 millimeters to about 7 millimeters.

The device can be machined from a surgical metal, such as stainless steel or another metal alloy that can sustain a corrosive environment.

The device can further be made from a molded material that withstands high temperatures without becoming brittle or cracking.

In an embodiment the device can include a non-slip coating disposed over the first end, such as a coating of sterilizable soft non-slip material, such as a synthetic rubber that can be between about 0.1 millimeters to about 0.5 millimeters in thickness.

In an operation on a patient, typically several trocars can be inserted into a patient through which suturing material is placed to approximate tissues. Using the knot pushing device, a surgeon can take a suture and passes it down the trocar through the two tissues and brings it back out through the same trocar. The surgeon can then make a knot that can then be slid down the trocar that can secure the stitch thereby securely apposing the two tissues together.

Suture material from one end of the formed stitch can then be passed through the hole of the device. The surgeon can then form a throw of a knot between the second end of the device and the trocar. While holding both ends of the suture together, the surgeon can push the throw with the planar face of the device down to the tissues through the trocar while holding onto the first end of the device until the loop is secure. The surgeon can then withdraw the device without taking it off the end of the suture, and make another throw, again pushing the newly formed throw into the patient proximal to the initial throw, forming a knot. The process can then be repeated until the surgeon determines that the tissues are secure.

A benefit of the invention is that it is a “staple-free” method of connecting tissues using standard sutures that more surgeons are familiar with.

FIG. 4 shows an embodiment of the planar face (16), with hole (20), a targeting light (31), and a camera (26) secured to the elongated body (10). A power supply (30), which can be remote is shown in this Figure, connected to the camera (26) and targeting light (31). An embodiment contemplates that the elongated body (10) can be hollow and cables or wires for the camera and the targeting light can run down the center of the elongated body.

Although the camera and/or targeting light can be connected on the outside of the elongated body, FIG. 4 shows an embodiment using a channel (32), which can be disposed along the axis of the elongated body (10) for containing the targeting light (31), the camera (26), cable (36), or combinations thereof.

The channel can have rounded edges where the channel meets the outside of the elongated body. The channel can have a slight lip (34) or overhang over the channel formed as an integral part of the body to contain a cable (36) in the channel. The cable can be fiber optic cables.

The camera (26) and/or the targeting light (31) can be in the channel, and the cable (36) can be removably affixed to the camera, the targeting light, or combinations thereof.

An embodiment of the device can contemplate that the first end can be removably secured to the elongated body.

An embodiment of the device can further contemplate that the second end can be removably secured to the elongated body.

An embodiment of the invention can contemplate that the first end can have a reduced slip grip for a doctor, which can be formed by etching, scoring, cutting with a laser, by sputtering or in some other manner.

An embodiment can further contemplate that a non-stick coating, such as Teflon™ can be disposed over all or a portion of the second end of the device, to enhance smooth insertion of throws into the body.

While these embodiments have been described with emphasis on the embodiments, it should be understood that within the scope of the appended claims, the embodiments might be practiced other than as specifically described herein.

What is claimed is:

1. A laparoscopic knot pushing device, comprising:
a. an elongated body having an axis, a first end and a second end, wherein the first end is adapted for gripping by a user;
b. a planar face formed in the second end wherein the planar face is integral with the elongated body, and wherein the planar face has a first side with a first rounded edge and a second rounded edge, and wherein the planar face is positioned 5 degrees to 20 degrees from the axis of the elongated body; and
c. a hole with a top rounded edge and a bottom rounded edge disposed in the planar face aligned with the elongated body axis.

2. The laparoscopic knot pushing device of claim 1, wherein the planar face is an integral portion of the elongated body forming a one-piece unit.

3. The laparoscopic knot pushing device of claim 1, wherein the planar face removably engages the elongated body.

4. The laparoscopic knot pushing device of claim 1, wherein the planar face has a width up to 9 millimeters and the hole in the planar face comprises a diameter between 1.5 millimeters to 7 millimeters.

5. The laparoscopic knot pushing device of claim 1, wherein the elongated body has a diameter between 3 millimeters to 9 millimeters.

6. The laparoscopic knot pushing device of claim 1, wherein the elongated body is hollow.

7. The laparoscopic knot pushing device of claim 1, wherein the elongated body and planar face comprises surgical grade stainless steel or a sterilizable, non-deformable, impact resistant, stiff with some flexibility, polymer, graphite composite or a polymer with elastomer blend.

8. The laparoscopic knot pushing device of claim 1, wherein the elongated body and planar face comprise an autoclavable non-deformable polymer disposed over a metal cylindrical body.

9. The laparoscopic knot pushing device of claim 1, wherein the elongated body is generally rounded.

10. The laparoscopic knot pushing device of claim 1, wherein the elongated body is tapered from a larger diameter at the first end to a smaller diameter at the second end.

11. The laparoscopic knot pushing device of claim 1, wherein the first end is removably secured to the elongated body.

12. The laparoscopic knot pushing device of claim 1, wherein the first end is scored for providing a more secure grip for the user.

13. The laparoscopic knot pushing device of claim 1, wherein the planar face is tapered to a diameter between 20 percent to 80 percent less than the diameter of the elongated body.

14. The laparoscopic knot pushing device of claim 1, wherein at least a portion of the first side of the planar face has a first rounded edge and a second rounded edge.

15. The laparoscopic knot pushing device of claim 1, wherein a non-stick coating is disposed over the second end.

16. An interchangeable laparoscopic knot pushing device comprising:
a. an elongated body having an axis, a removable first end and a second end, wherein the first end is a holding portion;
b. a planar face removably secured to the second end, wherein the planar face connects to the elongated body, and wherein the planar face has a first side with a first rounded edge and a second rounded edge and wherein the planar face is positioned 5 degrees to 20 degrees from the axis of the elongated body;
c. a hole with a top rounded edge and a bottom rounded edge disposed in the planar face aligned with the elongated body axis; and
d. a camera disposed in communication with the second end, and wherein the camera is adapted to removably engage the elongated body when the planar face is removed from the elongated body.

17. The interchangeable laparoscopic knot pushing device of claim 16, further comprising: a targeting light disposed in communication with the second end and connected to a power supply, wherein the targeting light is adapted to be removable from the elongated body.

18. The interchangeable laparoscopic knot pushing device of claim 16, wherein the elongated body is hollow for supporting cables and wires from the camera to a power supply.

19. The interchangeable laparoscopic knot pushing device of claim 16, further comprises a channel disposed along the axis of the elongated body for containing the targeting light, the camera, cables, or combinations thereof.

20. An interchangeable laparoscopic knot pushing device comprising:
a. an elongated body having an axis, a removable first end and a second end, wherein the first end is a holding portion;
b. a planar face removably secured to the second end, wherein the planar face connects to the elongated body, and wherein the planar face has a first side with a first rounded edge and a second rounded edge and wherein the planar face is positioned 5 degrees to 20 degrees from the axis of the elongated body;
c. a hole with a top rounded edge and a bottom rounded edge disposed in the planar face aligned with the axis to the elongated body axis; and
d. a targeting light disposed in communication with the second end, and wherein the targeting light removably engages the elongated body.
